(19) United States
(12) Patent Application Publication
(10) Pub. No.: US 2020/0327970 A1
(43) Pub. Date: Oct. 15, 2020

(54) ADDENDUM-BASED REPORT QUALITY SCORECARD GENERATION

(71) Applicant: KONINKLIJKE PHILIPS N.V., EINDHOVEN (NL)

(72) Inventor: Merlijn SEVENSTER, HAARLEM (NL)

(21) Appl. No.: 16/753,504

(22) PCT Filed: Oct. 8, 2018
(86) PCT No.: PCT/EP2018/077285

§ 371 Date: Apr. 3, 2020

Related U.S. Application Data
(60) Provisional application No. 62/568,836, filed on Oct. 6, 2017.

(51) Int. Cl. G16H 15/00; G16H 30/40; G06F 40/194; G06K 9/62

(52) U.S. Cl. CPC G16H 15/00; G06K 9/6267; G06F 40/194; G16H 30/40

(57) ABSTRACT

The following relates to medical equipment technology, and in particular to radiology report technology. In one embodiment, an original radiological report and an addended radiological report are received. The original radiological report and the addended radiological report may be part of the same or separate documents. The original radiological report and the addended radiological report may be compared. Each difference may be classified and scored. A display device may be controlled to display at least one of the scores.

### BACKGROUND

The following relates to the medical arts, radiology arts, and related arts, and in particular to radiology reporting technology.

Various commercial tools exist for providing performance assessments for medical institutions, clinical departments, and other facets of medical care institutions. For example, PerformanceBridge Solutions (available from Koninklijke Philips N.V., Eindhoven, the Netherlands) provides expert services, data analytics tools, and the like for assessing and improving clinical workflow.

In this commercial field, there is a need for providing more probative assessment of the quality of radiology reports issued by a Radiology Department. However, such assessment is challenging because of the highly specialized nature of radiology reading, which limits the usefulness of conventional benchmarks such as throughput and qualitative peer review. To the contrary, quality assessments for radiology reports should ideally be performed by radiologists, who have the highly specialized expertise necessary to provide a meaningful evaluation. However, it can be difficult to effectively employ radiologists in such a quality review role. One issue is the adverse impact to cost and efficiency when skilled radiologists are diverted from productive radiology reading other patient care-related tasks to perform the ancillary role of quality review. Another potential difficulty is that a radiologist may be unwilling to criticize another radiologist working in the same department. Use of radiologists hired from outside on a contractual basis could mitigate this latter difficulty, but would still involve higher costs.

### SUMMARY

In accordance with one aspect, a system for improving processing of a radiological report includes one or more electronic processors configured to: retrieve an original radiological report from a database; retrieve an addended radiological report corresponding to the original radiological report from the database; compare the original radiological report with the addended radiological report to determine one or more differences between the original radiological report and the addended radiological report; classify each difference of the one or more differences by assigning a class to each difference; assign a score for each difference based on the class assigned to the difference, the score grading severity of an error or omission in the original radiological report indicated by the difference; and control a display a quality assessment score for the original radiology report computed using device to display at least one of the scores.

The system as described in the preceding paragraph may further include that the one or more processors are further configured to receive the original radiological report and the addended radiological report as separate documents. The system may further include that the one or more processors are further configured to receive the original radiological report and the addended radiological report as a single document. The one or more processors may further be configured to apply a natural language processing algorithm to separate the original radiological report from the addended radiological report. The one or more processors may further be configured to assign the score for each difference further based on contextual parameters, the contextual parameters comprising one or more of: a time difference between a finalization time of the original radiological report and a finalization time of the addended radiological report; whether the report was stat; and whether the addended radiological report was created by an author of the original report. The one or more processors may further be configured to: if the one or more differences comprises more than one difference, create the quality assessment score for the original radiological report as the assigned score which grades a highest severity of an error or omission in the original radiological report. The one or more processors may further be configured to: control the display device to display section-specific views, the section-specific views including: an abdomen-specific view; a thoracic-specific view; and a neuro-specific view; and control the display device to display seniority-specific views, the seniority-specific views including: a resident-specific view; a fellow-specific view; and a senior attending-specific view. The one or more processors may further be configured to classify each difference by: counting a number of detected keywords, and a number of detected phrases; and assigning the class to each difference based on the number of detected keywords and the number of detected phrases.

In accordance with another aspect, a method, performed by one or more electronic processors, for improving processing of a radiological report includes: retrieving an original radiological report from a database; retrieving an addended radiological report corresponding to the original radiological report from the database; comparing the original radiological report with the addended radiological report to determine one or more differences between the original radiological report and the addended radiological report; classifying each difference of the one or more differences by assigning a class to each difference; assigning a score for each difference based on the class assigned to the difference, the score grading severity of an error or omission in the original radiological report indicated by the difference; and controlling a display device to display a quality assessment score for the original radiology report computed using at least one of the scores.

The method as described in the preceding paragraph may further include that the original radiological report and the addended radiological report are received as separate documents. The method may further include that the original radiological report and the addended radiological report are received as a single document in which the one or more differences between the original radiological report and the addended radiological report are indicated by annotations to the single document. The method may further include applying a natural language processing algorithm to separate the original radiological report from the addended radiological report. The method may further include that scores are assigned further based on contextual parameters, the contextual parameters comprising: a time difference between a finalization time of the original radiological report and a finalization time of the addended radiological report; whether the report was stat; and whether the addended radiological report was created by an author of the original report. The method may further include: in response to the one or more differences comprising more than one difference, creating a quality assessment score for the original radiological report (100) as the assigned score which grades a highest severity of an error or omission in the original radiological report.

The method may further include: controlling the display device to display section-specific views, the section-specific views including: an abdomen-specific view; a thoracic-specific view; and a neuro-specific view; and controlling the display device to display seniority-specific views, the seniority-specific views including: a resident-specific view; a fellow-specific view; and a senior attending-specific view. The method may further include: counting a number of detected keywords, and a number of detected phrases; and assigning the class to each difference based on the number of detected keywords and the number of detected phrases.

In accordance with yet another aspect, a system for improving processing of a radiological report includes a change detection engine configured to: receive an original radiological report; receive an addended radiological report corresponding to the original radiological report; and compare the original radiological report with the addended radiological report to determine one or more differences between the original radiological report and the addended radiological report. The system further includes a classification engine configured to classify each difference of the one or more differences by assigning a class to each difference. The system still further includes a severity determination engine configured to score each difference based on the class assigned to the difference. The system still further includes a scorecard determination engine configured to control a display device to display at least one of the scores.

The system as described in the preceding paragraph may further include that the change detection engine is further configured to receive the original radiological report and the addended radiological report as separate documents. The system may further include that the change detection engine is further configured to receive the original radiological report and the addended radiological report as a single document. The system may further include that the change detection engine is further configured to apply a natural language processing algorithm to separate the original radiological report from the addended radiological report.

The invention may take form in various components and arrangements of components, and in various steps and arrangements of steps. The drawings are only for purposes of illustrating the preferred embodiments and are not to be construed as limiting the invention.

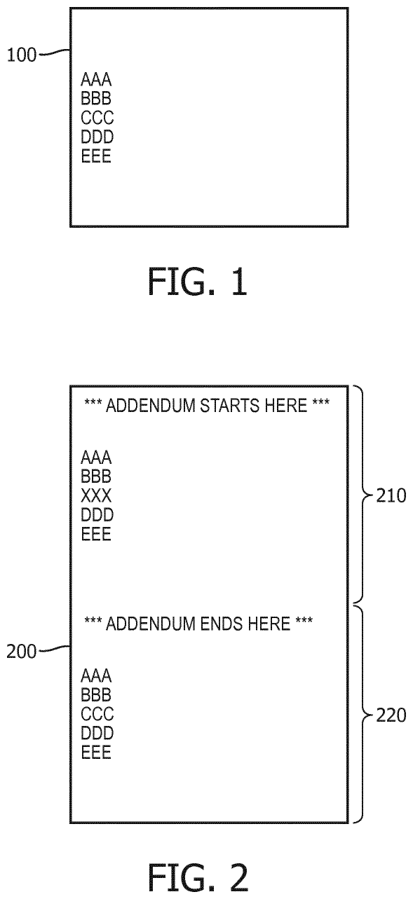

FIG. 1 illustrates an example of an original radiological report.

FIG. 2 illustrates an example of an addended radiological report.

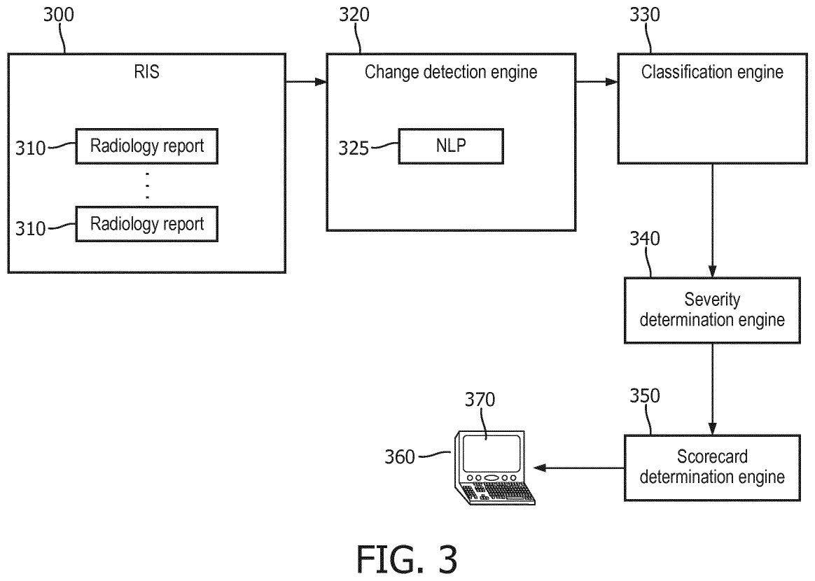

FIG. 3 diagrammatically shows a preferred embodiment.

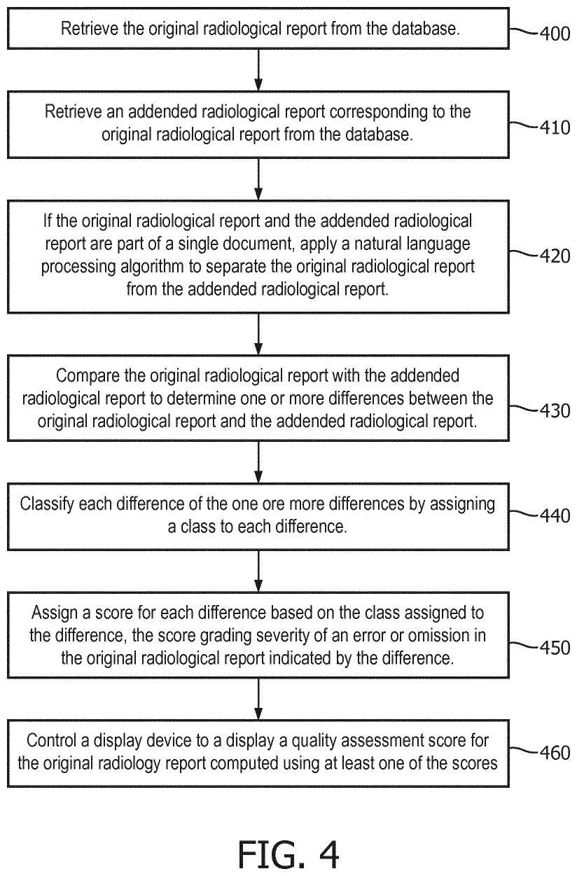

FIG. 4 diagrammatically shows a preferred embodiment of a method.

### DETAILED DESCRIPTION

The end product of a radiology interpretation (also referred to as a radiology reading) is a radiology report, which is typically an entirely or primarily free-text document stating findings and main conclusions. In practice, there is vast variability between radiology reports in terms of quality. It is thus difficult to make the concept of report quality objective and quantifiable. The approaches described herein leverage the insight that consequential report errors and omissions are corrected through the use of addenda/addendum. For example, approaches described include capturing addendum changes, categorizing them and preparing a scorecard based on this analysis.

It is relatively common that an initially issued radiology report may be modified at a later date. This may be done to correct typographical errors, or to correct more serious problems such as a missed finding or the even more serious problem of an erroneous finding. In another scenario, the radiology report may be later modified to incorporate additional information that was not available at the time of the initial reading. For example, biopsy results may be added when they become available to provide a more self-contained radiology report. Thus, the mere presence of a modification to the original report does not, by itself, indicate that the original radiology report contained an error. To account for this, in quality assessment embodiments disclosed herein, differences (i.e. addenda) between the addended report and the original report are classified into classes of a set of classes, for example including classes representing typographical corrections or addition of ancillary material (these are differences that do not indicate substantive problems with the report), classes representing omitted or erroneous benign findings (in an oncology setting, these are differences which are more severe, but still do not strongly impact the clinical value of the report), or classes representing omitted or erroneous malignant findings (these are differences that are most severe in an oncology setting as they can result in misdiagnosis or similar clinical errors). The quality assessment then scores each difference based on its class (and, in some embodiments, based on other information) to grade the severity of the error or omission in the original radiological report indicated by the difference. (Note that in some situations, such as an addendum that adds ancillary material that was unavailable at the time the original radiology report was drafted, the score may grade the severity as “null”, i.e. as not representing an error or omission at all).

In existing practice, the modification is implemented by way of an addendum. More specifically, to maintain the integrity of medical record-keeping, by way of the addendum: the original radiology report is preserved in the Radiology Information System (RIS) or Picture Archiving and Communication System (PACS), and a new document (or document version) is created which includes the modification, preferably flagged by standard notation such as “ADDENDUM STARTS HERE” . . . “ADDENDUM ENDS HERE.” Retention of the original radiology report serves various purposes, such as providing an auditable history of the radiology examination, and possibly compliance with medical records retention policies and/or governmental regulations.

More generally, in current medical practice, focus is shifting from volume to value, and thus new metrics are being developed to quantify the value-add of care providers. This shift is particularly disruptive for radiologists and radiology departments, as they essentially provide a service to the referring physician that could be provided by another radiologist or another radiology department.

The approaches described herein solve many problems including that it is hard to make the concept of report quality objective and quantifiable. One possible approach might focus on the use of hedging language, which is intentional but inappropriate use of vague and inconclusive phrases, or might attempt to assess the completeness of recommendations. However, there are cases where vague and inconclusive language is appropriate and incomplete recommendations are as helpful as complete ones. In other words, report quality is highly dependent on the larger context, which in itself is hard, if not impossible, to formalize for the sake of assessing the quality of an individual report.

The approaches described herein leverage the insight that consequential report errors and omissions are corrected through the use of addenda. These addenda are input by radiologists during the course of their normal productive radiology readings or other patient care-related tasks, and hence the addenda are available without imposing additional workload on departmental radiologists. By leveraging these addenda, the disclosed approaches provide quality assessments from radiologists without affirmatively burdening the radiologists with performing such assessments. Methods are disclosed that capture addendum changes, categorize them and prepare a scorecard based on this analysis.

Once a report is addended, a new report is created that contains two verbatim copies of the original report separated by an addendum header and footer. The radiologist then adjusts the language in one copy while leaving the other copy intact for reference by the referring physician. For instance, FIG. 1 shows original report 100. If the original report 100 is addended by revising the line “CCC” to “XXX,” then an addended radiological report 200 would be created as shown in FIG. 2. In the example of FIG. 2, the addended report 200 includes both addendum 210 and an original document 220.

Broadly, the approaches disclosed herein leverage addendums for quality control assessment. Each addendum is detected, and the original and addendum radiology reports are compared to identify the modification (which may, in general, be added material, edited material, or deleted material, or some combination of these). The modification is classified as to its type based on keywords, the type of modification (e.g. addition, deletion, grammatical editing, word-level editing, or so forth), or other features of the modification. As examples, a given modification may be classified as “typographic correction”, “missed measurement,” “added ancillary clinical data,” “missed benign finding,” “missed potentially malignant finding,” “missed correlation with pathology outcome,” or so forth. Each modification is assigned a severity score based on its classification. Optionally, other information such as the identity of the person making the modification may be used to adjust the class-based score up or down. For example, if the head of the Radiology Department made the modification, this may merit adjusting the severity score upward on the rationale that the department head would only perform such a modification to correct a serious mistake. If an addendum report includes more than one modification, then the score for the addendum report may be taken as the most significant (e.g. the highest) severity score of the plural modifications. In this way the overall score indicates the most severe error in the report. The resulting severity scores may be aggregated by radiologist, or by work shift, or on the basis of other criteria in order to generate actionable data analytics for purposes such as radiology personnel assessment, training, or so forth.

With reference to FIG. 3, Radiology Information System (RIS) 300 stores radiology reports 310, which include both original reports and addended reports. The RIS 300 may be embodied as a network-based database or so forth. While “RIS” is a commonly used term for a database storing radiology reports and other radiology-related data, the radiology database may be otherwise-termed, e.g. referred to as a Picture Archiving and Communication Service (PACS) or by some other nomenclature. Change detection engine 320 is an engine that can take an addended report and detect changes relative to the original. More particularly, the change detection engine 320 consumes an addended report and its original. In one example (scenario A), the change detection engine 320 operates on a single document that contains both an addendum and an original report, such as in the example of FIG. 2. In another example (scenario B), the addendum and the original report are two physically different documents, for example stored as different versions of the radiology report.

In scenario A, a natural language processing engine (NLP) 325 may be used to separate the addended version from the original document. The NLP 325 can be based on detecting the default addendum headers and footers. This reduces scenario A to scenario B, and thus this disclosure may refer to the addendum as physically separated from the original.

String matching techniques may be used to find changes between the original report and the addendum. Particularly, the Levenshtein difference algorithm was found to be advantageous in this regard. The Levenshtein difference algorithm can be used to convert the one report into the other using a set of syntactic operations. As the document is being converted, it can be tracked which portions of the report are changes and which portions remain static. For instance, in the example of FIGS. 1 and 2, the input strings are as follows:

AAA\nBBB\nCCC\n\DDD\nEEE

AAA\nBBB\nXXX\nDDD\nEEE

In this example, the strings CCC and XXX fall out as the report portions that are changed. Note that it is possible that either CCC or XXX is empty, if only text was added or removed from the original report, respectively. More generally, other types of “track changes” algorithms may be employed to detect the differences between the addended report compared with the original report.

In an advanced embodiment, a report segmentation tool is used to recognize report section and sentence ends. In this embodiment, the sentences containing the report changes can be retrieved instead of the revised text elements (which may not be entire sentences) and labeled with the section type from which they originate (e.g., Findings, Conclusions).

Returning to FIG. 3, classification engine 330 is an engine that can categorize the changes found by the change detection engine 320 into two or more categories. The classification engine 330 receives the changed text fragments (or sentences containing them) from the change detection engine 320, of which there can be one or more changes (e.g., CCC to XXX and DDD to YYY, etc.).

Two or more pre-determined change categories may be used, for instance “missed benign finding,” “missed potentially malignant finding,” “correlation with pathology outcome,” “typographic error,” “missing measurement,” and so forth.

The classification engine 330 may, in one embodiment, map each revision (e.g. CCC to XXX) onto one of the pre-determined revision categories (i.e. a set of classes). In one implementation, a list of keywords or common phrases is used associated with each categories. Using semantic techniques, this list can be extended by adding known synonyms using a background ontology, a standard dictionary or unsupervised learning techniques (e.g. “word2vec”). Using matching techniques accounting for common lexical variations (e.g., through stemming), the text fragments can be searched for the list of keywords and common phrases. The detected keywords and phrases can be used to assign the class. For instance, the classification engine 330 can count the number of detected keywords and phrases per category and assign the category that has the most hits. As another example, quantitative value indications such numerical values, standard units of length or volume (e.g. “cm”) or so forth may be leveraged in classifying a difference as an addition or modification of a measurement. In another implementation, the classification is based on machine learning that uses the extracted keywords and phrases as features and optimally predicts the final category. This implementation, although likely more accurate, will require a (manually curated) ground truth.

Optionally, other information may be used in classifying a difference. For example, if the radiology report is semi-structured, with different defined sections for different types of information (such as a patient data section, a findings section, a conclusions section) then the difference may be classified in part based on which section of the report in which it occurs.

The classification engine 330 can also cycle through each string or segment until of a radiology report until each string or segment of the radiology report is classified before moving on to the next radiology report.

Severity determination engine 340 is an engine that can determine the severity of a change based on the changed language and optionally further based on contextual parameters. In general, a score is assigned for each difference based on the class assigned to the difference (and optionally based on further information). The score grades severity of an error or omission in the original radiological report indicated by the difference. For example, in one possible grading scheme any difference assigned to a class representing addition or modification of a potentially malignant finding is assigned a score that grades higher severity than any difference assigned to a class representing addition or modification of a benign finding. Further, any difference assigned to a class representing addition or modification of a finding is assigned a score that grades higher than any difference assigned to a class representing a typographical correction. As yet another possible scoring rule, any difference assigned to a class representing a typographical correction may score higher than any difference assigned to a class representing addition of clinical data unavailable at the time the original report was prepared (since this lattermost change does not reflect any error at all in the original radiology report). These are merely examples, and the classes and scoring may be designed with varying levels of granularity and clinical domain-specificity depending upon the desired characteristics of quality assurance assessment. For example, classes related to findings may be further refined by the type of finding (e.g. malignant tumor versus bone fracture versus cardiac abnormality and so forth), typographical corrections may be refined on the basis of the type of error (e.g., an error in patient name may be scored to be more severe than a misspelled word), and/or so forth. In one implementation, the severity determination engine 340 analyses the severity of a change on a standardized scale. In one example, the severity determination engine 340 uses a weighing scheme that leverages the category predicted by the classification engine 330 as well as contextual parameters such as time between finalization of original and addended report; whether the report was stat; whether the addendum was created by the author of the original report; etc. Each contextual parameter can be associated with a specific severity weight, which can be added up to obtain the sum total severity. For instance, “missed benign finding” may have 1 severity weight, whereas “missing measurement” has 3 severity weight; if the addendum was issued within 1 hour of finalization of the original, this might add 1 severity weight, otherwise 5 could be added. The sum total severity can be used as is or mapped onto a standardized Likert scale, e.g., Mild/Moderate/Severe. If there is more than one change in the addendum, the severity determination engine 340 can take the change with highest severity.

The scorecard generation engine 350 is an engine that can generate a quality scorecard based on addendum analysis. In general, a quality assessment score for the original radiology report (100) is computed using at least one of the scores assigned for differences between the addended and original reports. If there is only one difference, then the score for that difference generally serves as the quality assessment score for the original report. If there are multiple differences (i.e. multiple addenda) then the assigned score which grades a highest severity of an error or omission in the original radiological report is set as the quality assessment score. On the other hand, if a report has no addenda, this may result in a “best” quality assessment score for the original (and in this case only) radiology report. In one implementation, the scorecard generation engine 350 accumulates the severity scores of all changes and presents them as a scorecard on various levels of granularity. For instance, a department-wide scorecard can be created assessing the distribution over the various severity categories. Similarly, a section-specific (e.g., abdomen, thoracic, neuro), seniority-specific (e.g., resident, fellow, junior attending, senior attending) and personalized view can be generated. The view can be such that individual addendum cases can be reviewed. The scorecard can be used as a mechanism to track improvements in report quality.

The classes of differences may also be usefully tabulated, e.g. for all radiology reports produced by a particular radiologist, so as to provide information on the type of errors or omissions that particular radiologist is prone to making. This can be useful feedback for the radiologist to improve his or her subsequent reporting practices. Similar tabulation of classes of differences can be made on a section level, workshift level, department level, and/or so forth, in order to identify and remediate institutional level reporting deficiencies.

The scorecard generation engine 350 may control a display device 370 of a computing device 360 to display the views and scores.

FIG. 4 illustrates a preferred embodiment of a first method. With reference thereto, in step 400, the original radiological report is received. In step 410, an addended radiological report corresponding to the original radiological report is received. In step 420, if the original radiological report and the addended radiological report are part of a single document, a natural language processing algorithm is applied to separate the original radiological report from the addended radiological report. In step 430, the original radiological report with the addended radiological report are compared to determine one or more differences between the original radiological report and the addended radiological report. In step 440, each difference of the one or more differences is classified by assigning a class to each difference. In step 450, each difference is scored based on the class assigned to the difference. In step 460, a display device is controlled to display at least one of the scores.

With reference back to FIG. 1, the disclosed data processing components, e.g. the change detection engine 320, classification engine 330, severity determination engine 340, and scorecard determination engine 350, are suitably implemented in the form of one or more electronic processors 360 executing instructions read from a non-transitory storage medium such as a hard drive or other magnetic storage medium, an optical disk or other optical storage medium, a cloud-based storage medium such as a RAID disk array, flash memory or other non-volatile electronic storage medium, or so forth. While the illustrative one or more electronic processors comprise an illustrative computer 360, more generally the one or more electronic processors may be a standalone desktop computer, or a network-based server computer, or a plurality of computers connected via an electronic network (e.g. WiFi, Ethernet, Internet, various combinations thereof, or so forth) to form a parallel computing resource, ad hoc cloud computing resource, or so forth.

It will be further appreciated that the data processing components, e.g. the change detection engine 320, classification engine 330, severity determination engine 340, and scorecard determination engine 350, disclosed herein may be embodied by a non-transitory storage medium storing instructions readable and executable by an electronic data processing device (such as the illustrative computer 360 or a computer server, a cloud computing resource or so forth) to perform the disclosed techniques. Such a non-transitory storage medium may comprise a hard drive or other magnetic storage medium, an optical disk or other optical storage medium, a cloud-based storage medium such as a RAID disk array, flash memory or other non-volatile electronic storage medium, or so forth.

Of course, modifications and alterations will occur to others upon reading and understanding the preceding description. It is intended that the invention be construed as including all such modifications and alterations insofar as they come within the scope of the appended claims or the equivalents thereof.

1. A system for improving processing of a radiological report, the system comprising:
one or more electronic processors configured to:
retrieve an original radiological report from a database;
retrieve an addended radiological report corresponding to the original radiological report from the database;
compare the original radiological report with the addended radiological report to determine one or more differences between the original radiological report and the addended radiological report;
classify each difference of the one or more differences by assigning a class to each difference;
assign a score for each difference based on the class assigned to the difference and further based on contextual parameters, the score grading severity of an error or omission in the original radiological report indicated by the difference; and
control a display device to display a quality assessment score for the original radiology report computed using at least one of the scores.

2. The system of claim 1, wherein the one or more electronic processors are configured to retrieve the original radiological report and the addended radiological report as separate documents.

3. The system of claim 1, wherein the one or more electronic processors are configured to retrieve the original radiological report and the addended radiological report as a single document in which the one or more differences between the original radiological report and the addended radiological report are indicated by annotations to the single document.

4. The system of claim 3, wherein the one or more electronic processors are further configured to apply a natural language processing algorithm to separate the original radiological report from the addended radiological report.

5. The system according to claim 1, wherein the contextual parameters comprise one or more of:
a time difference between a finalization time of the original radiological report and a finalization time of the addended radiological report;
whether the report was stat; and
whether the addended radiological report was created by an author of the original report.

6. The system according to claim 1, wherein the one or more electronic processors are further configured to:
if the one or more differences comprises more than one difference, create the quality assessment score for the original radiological report as the assigned score which grades a highest severity of an error or omission in the original radiological report.

7. The system according to claim 1, wherein the one or more electronic processors are further configured to:
control the display device to display section-specific views, the section-specific views including:
an abdomen-specific view;
a thoracic-specific view; and
a neuro-specific view; and
control the display device to display seniority-specific views, the seniority-specific views including:
a resident-specific view;
a fellow-specific view; and
a senior attending-specific view.

8. The system according to claim 1, wherein the one or more electronic processors are configured to classify each difference by:
counting a number of detected keywords, and a number of detected phrases; and
assigning the class to each difference based on the number of detected keywords and the number of detected phrases.

9. The system according to claim 1, wherein the one or more electronic processors are further configured to classify each difference as belonging to a class which is a member of a set of classes that include:
at least one class representing typographical corrections;
at least one class representing addition or modification of a measurement; and
at least one class representing addition or modification of a radiology finding.

10. The system according to claim 9, wherein the at least one class representing addition or modification of a radiology finding includes:
at least one class representing addition or modification of a benign finding; and
at least one class representing addition or modification of a potentially malignant finding;
wherein any difference assigned to the at least one class representing addition or modification of a potentially malignant finding is assigned a score that grades higher severity than any difference assigned to the at least one class representing addition or modification of a benign finding.

11. A method, performed by one or more electronic processors, for improving processing of a radiological report, the method comprising:
retrieving an original radiological report from a database;
retrieving an addended radiological report corresponding to the original radiological report from the database;
comparing the original radiological report with the addended radiological report to determine one or more differences between the original radiological report and the addended radiological report;
classifying each difference of the one or more differences by assigning a class to each difference;
assigning a score for each difference based on the class assigned to the difference and further based on contextual parameters, the score grading severity of an error or omission in the original radiological report indicated by the difference; and
controlling a display device to display a quality assessment score for the original radiology report computed using at least one of the scores.

12. The method of claim 11, wherein the original radiological report and the addended radiological report are retrieved as separate documents.

13. The method of claim 11, wherein the original radiological report and the addended radiological report are retrieved as a single document in which the one or more differences between the original radiological report and the addended radiological report are indicated by annotations to the single document.

14. The method of claim 13, further comprising applying a natural language processing algorithm to separate the original radiological report from the addended radiological report.

15. The method according to claim 11, wherein the contextual parameters comprise
a time difference between a finalization time of the original radiological report and a finalization time of the addended radiological report;
whether the report was stat; and
whether the addended radiological report was created by an author of the original report.

16. The method according to claim 11, further comprising:
in response to the one or more differences comprising more than one difference, creating a quality assessment score for the original radiological report as the assigned score which grades a highest severity of an error or omission in the original radiological report.

17. The method according to claim 11, further comprising:
controlling the display device to display section-specific views, the section-specific views including:
an abdomen-specific view;
a thoracic-specific view; and
a neuro-specific view; and
controlling the display device to display seniority-specific views, the seniority-specific views including:
a resident-specific view;
a fellow-specific view; and
a senior attending-specific view.

18. The method according to claim 11, wherein each difference is classified by:
counting a number of detected keywords, and a number of detected phrases; and
assigning the class to each difference based on the number of detected keywords and the number of detected phrases.

19. A system for improving processing of a radiological report, the system comprising:
a change detection engine including one or more electronic processors configured to:
receive an original radiological report;
receive an addended radiological report corresponding to the original radiological report; and
compare the original radiological report with the addended radiological report to determine one or more differences between the original radiological report and the addended radiological report;
a classification engine including said one or more electronic processors configured to classify each difference of the one or more differences by assigning a class to each difference;
a severity determination engine including said one or more electronic processors configured to:
score each difference based on the class assigned to the difference and further based on contextual parameters; and
a scorecard determination engine including said one or more electronic processors configured to control a display device to display at least one of the scores;
wherein the contextual parameters comprise one or more of:
a time difference between a finalization time of the original radiological report and a finalization time of the addended radiological report;
whether the report was stat; and
whether the addended radiological report was created by an author of the original report.

20. The system of claim 19, wherein the change detection engine is further configured to receive the original radiological report and the addended radiological report as separate documents.
